(19) United States
(12) Patent Application Publication
(10) Pub. No.: US 2016/0032411 A1
(43) Pub. Date: Feb. 4, 2016

(54) SELECTIVE DETECTION OF HEPATITIS A, B, C, D, OR E VIRUSES OR COMBINATION THEREOF

(71) Applicant: THE USA, AS REPRESENTED BY THE SECRETARY, DEPART- MENT OF HEALTH AND HUMAN SERVICES, Atlanta, GA (US)

(72) Inventors: Maja Kodani, Stockbridge, GA (US); Tonya Mixson-Hayden, Lilburn, GA (US); Saleem Kamili, Lilburn, GA (US)

(21) Appl. No.: 14/777,353

(22) PCT Filed: Mar. 14, 2014
(86) PCT No.: PCT/US2014/029049

§ 371 Date: Sep. 15, 2015

Related U.S. Application Data
(60) Provisional application No. 61/792,293, filed on Mar. 15, 2013.

(51) Int. Cl. C12Q 1/70

(52) U.S. Cl. CPC C12Q 1/706

(57) ABSTRACT

Processes and compositions are provided for the detection of hepatitis viruses in a sample. Particular processes and compositions are provided for the selective detection of HDV. Also provided are processes and compositions for the simultaneous detection of two or more hepatitis viruses that for the first time provide rapid, reliable, and simple detection of any known hepatitis vims in a sample using a single set of reaction conditions.

### CROSS REFERENCE TO RELATED APPLICATIONS

This application depends from and claims priority to U.S. Provisional Application No. 61/792,293 filed Mar. 15, 2013 and U.S. Provisional Application No. 61/938,220 filed Feb. 11, 2014, the contents of each of which are incorporated herein by reference.

### GOVERNMENT INTEREST

The invention described herein may be manufactured, used, and licensed by or for the United States Government.

### FIELD OF THE INVENTION

This invention relates generally to processes for detection of virus in fluid samples. More specifically, the instant invention relates to selective detection of hepatitis D virus (HDV) in biological or other fluid media. In addition, the invention relates to selective and multiplex detection of hepatitis A, B, C, D, E, or any combination thereof. Processes are described for rapid and sensitive detection of hepatitis viruses in human and animal biological samples and quantification thereof. Diagnostic kits are provided for detection of one or more hepatitis viruses in a clinical, laboratory, or field setting.

### BACKGROUND OF THE INVENTION

To date five viruses have been etiologically associated with viral hepatitis. These five viruses are denoted hepatitis A virus (HAV), hepatitis B virus (HBV), hepatitis C virus (HCV), hepatitis delta virus (HDV) and hepatitis E virus (HEV). These viruses vary widely in their natural history, genome composition, and mode of transmission. HAV, HCV, HDV and HEV are RNA viruses while HBV is a DNA virus.

An estimated 4.4 million Americans live with chronic viral hepatitis, yet many are unaware of their infection status. Infections with hepatitis B, C and D viruses can lead to cirrhosis and liver cancer and in many cases liver transplantation is the only treatment to save a patient's life. HEV infection has been associated with a severe disease and death in pregnant women and chronic infections among immunocompromised individuals. Hepatitis viruses B, C, and D which are transmitted parenterally, can lead to chronic liver disease and hepatocellular carcinoma.

HDV is responsible for severe acute and chronic hepatitis in patients previously or co-infected with HBV. Treatment relies on long-term administration of high-doses of alpha-interferon (IFN) and its efficacy is usually followed by the detection of specific anti-HDV IgM or HDV genome in serum. Individuals with concurrent acute HBV and HDV infection are considered co-infected, whereas patients who are chronically infected with HBV may develop a superinfection with HDV. Co-infection or superinfection with HDV can cause more severe hepatitis than HBV infection alone, including more rapid progression to liver disease and higher mortality rates. Due to this risk, it is important for patients who are HBsAg positive to be tested for HDV infection.

Present methods for diagnosis of HDV infection, in particular, rely on the detection of specific anti-HDV antibodies, with the presence of anti-HDV IgM reflecting ongoing viral replication. However, this serological approach for the detection of virus replication suffers in part due to HDV antigen being rarely detected in the serum, except in the case of acute infection (prior to the antibody sero-conversion), or among severely immunosuppressed chronically infected patients. Although anti-HDV antibody status can assess whether an individual has been exposed to HDV, it cannot determine if the infection is active or resolved.

There are two forms of the HDAg, both coded by the same open reading frame (ORF). The small hepatitis D antigen (S-HDAg) is 195 amino acids long (24 kDa), while large hepatitis D antigen (L-HDAg) is 19 amino acids longer on the carboxyl-terminal end (27 kDa), consisting of a stretch of variable, yet genotype-specific, membrane-attaching sequence and serves as the virion assembly signal (Lai 2005). Assembly of the viral particle requires both viral proteins to form the ribonucleoprotein. In the course of viral replication, the ribonucleoprotein buds through the hepatocyte endoplasmic reticulum, and acquires an envelope in which the hepatitis B surface antigens (HBsAg) are embedded.

Extensive sequence analyses of numerous isolates have lead to the classification of HDV in at least 8 distinct clades with different geographic distributions. Genotype 1 is prevalent world-wide with other genotypes being endemic to different parts of the world. Genotype 2 is found in southeast Asia, Taiwan, China and Japan. Genotype 3 is endemic to the Amazon Basin. Genotype 4 is found in Taiwan and Japan. Finally, genotypes 5 to 8 are prevalent in Africa.

Since viral hepatitis caused by different hepatitides is clinically indistinguishable, various testing algorithms need to be employed to determine the exact etiology of infection. Nucleic acid testing (NAT) remains the gold standard for diagnosis of active and viremic stages of infection with any hepatitides. Several assays, including those approved by the Food and Drug Administration (FDA), are commercially available for the detection of HBV DNA and HCV RNA. However, a NAT-based multiplex assay that can detect all five hepatitis virus in the same format and under identical experimental conditions has not been developed. Reasons for this include the lack of a sufficient detection system specific of HDV as well as the inability to incorporate detection systems for the previously detectable viruses into a single assay suitable for multiplex and simultaneous detection.

Thus, there is a need for a rapid, sensitive, and discriminatory assay for detection of HDV or all five known viruses simultaneously in complex clinical or laboratory samples.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A first object of the invention is to provide a process for the rapid and specific detection of HDV in simple or complex sample. Processes that address this object include producing an amplification product by amplifying a hepatitis virus, optionally HDV nucleotide sequence using a forward primer that interacts with an HDV genome at positions 816-834, and a reverse primer, under conditions suitable for a polymerase chain reaction; and measuring the amplification product to detect HDV in said biological sample. In some embodiments, the forward primer includes at least two degenerate nucleotide positions. Optionally, a forward primer consists of the sequence of SEQ ID NO: 1. Optionally, the reverse primer binds a region of a HDV genome between nucleotides 894 to 908. Optionally, the reverse primer consists of the sequence of SEQ ID NO: 2. A process optionally used a probe that will bind a region of a HDV genome in the region of nucleotides 853 to 890. A probe optionally includes at least two positions of nucleotide degeneracy. Optionally, a probe consists of the nucleotide sequence of SEQ ID NO: 3. The use of a probe optionally includes the step of detecting a first detection signal from the probe hybridized to the amplification product. Optionally, any of the above processes in any combination is used to diagnose HDV infection in a subject.

The processes of detecting HDV optionally further include comparing the first detection signal to a second detection signal, where the second detection signal results from detection of a complementary amplification product produced from a sequence of HDV. Optionally, the second detection signal is generated in parallel with said first detection signal.

Optionally, the first detection signal is compared to a third detection signal from a nucleic acid calibrator extracted in parallel to the biological sample. Optionally, the nucleic acid calibrator comprises a known amount of HDV and a known amount of a medium similar to the biological sample.

In some embodiments, the process of any of the above or any combination of the above elements optionally also includes producing a second amplification product by amplifying a nucleotide sequence from one or more viruses selected from the group consisting of HAV, HBV, HCV, and HEV; and measuring said second amplification product to detect said virus in said biological sample. Optionally, the one or more viruses is or includes HAV and said step of producing is using one or more nucleotides selected from the group consisting of SEQ ID NO: 16, SEQ ID NO: 17, and SEQ ID NO: 18. Optionally, the one or more viruses is or includes HBV and said step of producing is using one or more nucleotides selected from the group consisting of SEQ ID NO: 19, SEQ ID NO: 20, and SEQ ID NO: 21. Optionally, the one or more viruses is or includes HCV and said step of producing is using one or more nucleotides selected from the group consisting of SEQ ID NO: 22, SEQ ID NO: 23, and SEQ ID NO: 24. Optionally, the one or more viruses is or includes HEV and said step of producing is using one or more nucleotides selected from the group consisting of SEQ ID NO: 25, SEQ ID NO: 26, and SEQ ID NO: 27.

It is another object of the invention to provide a process for the simultaneous detection of more than one hepatitis virus in a biological sample. Optionally, the hepatitis virus is one or more of the viruses selected from the group consisting of HAV, HBV, HCV, HDV, and HEV. In some embodiments, the process detects the presence or absence of HAV, HBV, HCV, HDV, and HDV, wherein the reaction conditions for said step of producing HAV, HBV, HCV, HDV, and HDV amplification products are identical. Optionally, a forward primer comprises a nucleotide sequence selected from the group consisting of SEQ ID NO: 1, SEQ ID NO: 16, SEQ ID NO: 19, SEQ ID NO: 22, SEQ ID NO: 25, and combinations of said primers. Optionally, a reverse primer comprises a nucleotide sequence selected from the group consisting of SEQ ID NO: 2, SEQ ID NO: 4, SEQ ID NO: 5, SEQ ID NO: 17, SEQ ID NO: 20, SEQ ID NO: 23, SEQ ID NO: 26, and combinations of said primers. Optionally, said step of measuring is using a probe comprising a nucleotide sequence selected from the group consisting of SEQ ID NO: 3, SEQ ID NO: 6, SEQ ID NO: 7, SEQ ID NO: 8, SEQ ID NO: 9, SEQ ID NO: 10, SEQ ID NO: 18, SEQ ID NO: 21, SEQ ID NO: 24, SEQ ID NO: 27, and combinations of said probes. The detection of one or more viruses in a biological sample optionally diagnoses hepatitis virus infection in a subject.

Another object of the invention is to provide a kit for the detection of one or more hepatitis viruses in a biological sample. A kit optionally includes materials suitable for the detection of one or more of HAV, HBV, HCV, HDV. HEV, or any combination thereof. A kit optionally includes a first forward primer, said first forward primer optionally interacts with an HDV genome at positions 816-834. A first forward primer optionally includes at least two degenerate nucleotides. Optionally, a forward primer consists of the sequence of SEQ ID NO: 1, SEQ ID NO: 16, SEQ ID NO: 19, SEQ ID NO: 22, or SEQ ID NO: 25, or combinations of said forward primers are included. A kit optionally includes a reverse primer. A reverse primer optionally binds a region of a HDV genome between nucleotides 894 to 908. A reverse primer optionally consists of the sequence of SEQ ID NOs: 2, 4, 5, 17, 20, 23, 26 or a combination of primers having at least one of SEQ ID NOs. 2, 4, 5, 17, 20, 23, or 26. A kit optionally further or on its own includes a probe that will bind a region of a HDV genome in the region of nucleotides 853 to 890. A probe optionally comprises the nucleotide sequence SEQ ID NO: 3, SEQ ID NO: 6, SEQ ID NO: 7, SEQ ID NO: 8, SEQ ID NO: 9, SEQ ID NO: 10, SEQ ID NO: 18, SEQ ID NO: 21, SEQ ID NO: 24, SEQ ID NO: 27, or the kit optionally includes any combination of said probes. In some embodiments, a kit includes one or more oligonucleotides comprising a nucleotide sequence selected from the group consisting of SEQ ID NO: 16, SEQ ID NO: 17. SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or combinations thereof.

It is another object the invention to provide compositions suitable for the detection of the presence or absence of one or more hepatitis viruses in a sample. A composition includes an oligonucleotide that comprises or consists of the sequence of SEQ ID NO: 1, SEQ ID NO: 2, SEQ ID NO: 3, SEQ ID NO: 4, SEQ ID NO: 5. SEQ ID NO: 6, SEQ ID NO: 7, SEQ ID NO: 8, SEQ ID NO: 9, SEQ ID NO: 10, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 27.

### BRIEF DESCRIPTION OF THE DRAWINGS

FIG. 1 represents an alignment of the qRT-PCR amplicon region of HDV using sequences representing all HDV genotypes;

FIG. 2 represents HDV qRT-PCR Assay Characteristics where positive control transcript preparation was prepared in 10-fold serial dilutions and A) is the linearity of the assay spans six logs and B) is the standard curve shows the efficiency of over 90%, with the slope of −3.65 and the R² value of 0.9907; and

FIG. 3 illustrates a TaqMan Array Card (TAC) workflow compared to the individual real-time PCR samples for 384 reactions, the number equivalent to one card.

### DETAILED DESCRIPTION OF THE PREFERRED EMBODIMENTS

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art. It is to be understood that the present invention is not limited to particular embodiments described, which may, of course, vary.

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Scientific and technical terms used herein are intended to have the meanings commonly understood by those of ordinary skill in the art. Such terms are found defined and used in context in various standard references illustratively including in part J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Wild, D., The Immunoassay Handbook, 3rd Ed., Elsevier Science, 2005; Gosling, J. P., Immunoassays: A Practical Approach, Practical Approach Series, Oxford University Press, 2005; Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley. 2002; J. D. Pound (Ed.) Immunochemical Protocols, Methods in Molecular Biology, Humana Press; 2nd ed., 1998; B. K. C. Lo (Ed.), and Antibody Engineering: Methods and Protocols, Methods in Molecular Biology, Humana Press, 2003, the contents of each of which are incorporated herein by reference.

The above difficulties in detection and quantification of hepatitis viruses in complex biological samples such at plasma, serum, or other are addressed by the present processes and materials. The invention has utility to detect the presence or absence of HDV in a biological sample either alone or in concert with additional methods for detection of other hepatitis viruses including HAV, HBV, HCV, HEV, and new viruses to be discovered. Also provided are materials and processes for the simultaneous detection of HAV, HBV, HCV, HDV, and HEV.

Several inventions are provided. First, are provided processes and materials such as primers and probes for the detection of HDV. Second, are provided processes and materials for the simultaneous detection of all two or more, optionally all five, hepatitis viruses that optionally employ the inventive primers and probes for HDV detection.

The following definitional terms are used throughout the specification without regard to placement relative to these terms.

As used herein, the term “variant” defines either a naturally occurring genetic mutant of HDV or a recombinantly prepared variation of HDV, each of which contain one or more mutations in its genome compared to the HDV of HDV genotype 1 (accession no. AF098261). The term “variant” may also refer to either a naturally occurring variation of a given peptide or a recombinantly prepared variation of a given peptide or protein in which one or more amino acid residues have been modified by amino acid substitution, addition, or deletion.

As used herein, the term “analog” in the context of a non-proteinaceous analog defines a second organic or inorganic molecule that possesses a similar or identical function as a first organic or inorganic molecule and is structurally similar to the first organic or inorganic molecule. Structural similarity includes the presence of one or more degenerate nucleotides, one or more nucleotides on either or both ends of the molecule, the presence of a locked nucleic acid, or other similarity.

As used herein, the term “derivative” in the context of a non-proteinaceous derivative defines a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated. A derivative also defined as a degenerate base mimicking a C/T mix such as that from Glen Research Corporation, Sterling, Va., illustratively LNA-dA or LNA-dT, or other nucleotide modification known in the art or otherwise.

As used herein, the term “mutant” defines the presence of mutations in the nucleotide sequence of an organism as compared to a wild-type organism.

A “purified” nucleic acid molecule is one that is separated from other nucleic acid molecules that are present in the natural source of the nucleic acid molecule and is often substantially free of other cellular material, or culture medium such as when produced by recombinant techniques, or substantially free of chemical precursors or other chemicals when chemically synthesized. This term is exclusive of a nucleic acid that is a member of a library that has not been purified away from other library clones containing other nucleic acid molecules.

As used herein, the term “hybridizes under stringent conditions” describes conditions for hybridization and washing under which nucleotide sequences having at least 30%, 35%, 40%, 45%, 50%, 55%, 60%, 65%, 70%, 75%, 80%, 85%, 90%, or 95% identity to each other typically remain hybridized to each other. Such hybridization conditions are described in, for example but not limited to, Current Protocols in Molecular Biology, John Wiley & Sons, N.Y. (1989), 6.3.1 6.3.6.; Basic Methods in Molecular Biology, Elsevier Science Publishing Co., Inc., N.Y. (1986), pp. 75-78, and 84-87; and Molecular Cloning. Cold Spring Harbor Laboratory, N.Y. (1982), pp. 387-389, and are well known to those skilled in the art. A preferred, non-limiting example of stringent hybridization conditions is hybridization in 6× sodium chloride/sodium citrate (SSC), 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at 50 to 65° C.

An “isolated” or “purified” nucleotide or oligonucleotide sequence is substantially free of cellular material or other contaminating proteins from the cell or tissue source from which the nucleotide is derived, or is substantially free of chemical precursors or other chemicals when chemically synthesized. The language “substantially free of cellular material” includes preparations of a nucleotide/oligonucleotide in which the nucleotide/oligonucleotide is separated from cellular components of the cells from which it is isolated or produced. Thus, a nucleotide/oligonucleotide that is substantially free of cellular material includes preparations of the nucleotide having less than about 30%, 20%, 10%, 5%, 2.5%, or 1% (by dry weight) of contaminating material. When nucleotide/oligonucleotide is produced by chemical synthesis, it is preferably substantially free of chemical precursors or other chemicals, i.e., it is separated from chemical precursors or other chemicals which are involved in the synthesis of the protein. Accordingly, such preparations of the nucleotide/oligonucleotide have less than about 30%, 20%, 10%, 5% (by dry weight) of chemical precursors or compounds other than the nucleotide/oligonucleotide of interest. In a preferred embodiment of the present invention, the nucleotide/oligonucleotide are isolated or purified.

As used herein, the term “isolated” virus or virus-like particle (VLP) is one which is separated from other organisms which are present in the natural source of the virus, e.g., biological material such as cells, blood, serum, plasma, saliva, urine, stool, sputum, nasopharyngeal aspirates, and so forth. The isolated virus or VLP can be used to infect a subject cell.

As used herein, the term “biological sample” is defined as sample obtained from a biological organism, a tissue, cell, cell culture medium, or any medium suitable for mimicking biological conditions, or from the environment. Non-limiting examples include, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, and vitreal fluid, and nasal secretions, throat or nasal materials. In a preferred embodiment, viral agents are contained in serum, whole blood, liver tissue, or other biological material believed to have HDV present or absent.

As used herein, the term “medium” refers to any liquid or fluid biological sample in the presence or absence of virus. Non-limiting examples include buffered saline solution, cell culture medium, acetonitrile, trifluoroacetic acid, combinations thereof, or any other fluid recognized in the art as suitable for combination with virus or cells, or for dilution of a biological sample or amplification product for analysis.

To determine the percent identity of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, nonlimiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used. Optionally, the MEGA5 software is used to align sequences. Tamura K, et al., Molecular Biology and Evolution, 2011; 28:2731-2739.

The percent identity between two sequences is determined using techniques similar to those described above, with or without allowing gaps. In calculating percent identity, typically only exact matches are counted.

As used herein, the terms “subject” and “patient” are synonymous and refer to a human or non-human animal, preferably a mammal including a human, non-primate such as cows, pigs, horses, goats, sheep, cats, dogs, avian species and rodents; and a non-human primate such as monkeys, chimpanzees, and apes; and a human, also denoted specifically as a “human subject.”

In some embodiments, inventive processes involve the use of real-time polymerase chain reactions (qRT-PCR). qRT-PCR is an accurate and sensitive assay of quantifying viral genomes, with the major advantage of avoiding post-PCR handling that can be source of DNA carryover. While RT-PCR has itself been suggested for detection of HDV RNA, the primers used in were designed on the basis of a region conserved among type 1, 2a and 2b HDV genotypes. These primers, however do not take in account the other HDV genotypes. Also, many primers suggested by prior methods, such as those in U.S. Patent Application No. 2010/0143894 fail to be capable of full PAN detection of all known HDV genotypes.

Perhaps more importantly, the prior primers and probes for HDV detection are incapable of use in a multiplex or simultaneous reaction with other primers and probes for detection of other hepatitis virus types. These problems are fully addressed by the presently provided primers and probes that: 1) do not suffer from negative secondary structure formation; 2) are used for PAN detection of all HDV genotypes; 3) are suitably matched in size, gc content, and melting temperature to be used as a component of a broader assay to detect all five hepatitis viral types; and 4) and are readily tailorable to additional detection of future HDV types. Moreover, the primers and probes also are capable of working with other assays for hepatitis virus infection due to their ability to function with samples extracted with a common extraction technique, function with the same PCR chemistry; and function under common cycling parameters. While HBV is a DNA virus, the rest are RNA viruses, so a universal nucleic acid extraction needs to be performed in order to amplify any of them from an infected individual. The primers and probes of the invention are specifically identified and produced to satisfy all these needs.

Some embodiments of the instant inventive process provide rapid, specific, and sensitive assay process for detection of HDV in biological samples by amplifying one or more nucleotide sequences with greater specificity to strains of HDV than HCV or other viral agents. A process optionally involves qRT-PCR methods. General principles of real time quantitative RT PCR are known in the Art and are for instance described in Poitras et al., Reviews in Biology and Biotechnolog, 2002; 2: 1-11 and Gibson et al., Genome Research, 1996; 6: 995-1001. Real-time PCR based on the TaqMan® technology allows DNA or cDNA quantification over a large dynamic range (10 to 10⁷ copies), and is therefore well adapted to the quantification of viral genomes. Moreover, the possibility of handling numerous samples and the absence of post-PCR handling makes it a safe and convenient approach for clinical diagnosis.

An oligonucleotide forward primer with a nucleotide sequence complementary to a unique sequence in an HDV nucleotide sequence illustratively in the region upstream from the HDAg coding region is hybridized to its complementary sequence and extended. Similarly, a reverse oligonucleotide primer complementary to a second strand of HDV nucleotide sequence in the same or an alternate HDV region is hybridized and extended. This system allows for amplification of specific gene sequences and is suitable for simultaneous or sequential detection systems.

The present invention relates to the use of the sequence information of HDV for diagnostic process. In particular, the present invention provides a process for detecting the presence or absence of nucleic acid molecules of HDV, natural or artificial variants, analogs, or derivatives thereof, in a biological sample. The process involves obtaining a biological sample from various sources and contacting the sample with a compound or an agent capable of detecting a nucleic acid sequence of HDV, natural or artificial variants, analogs, or derivatives thereof, such that the presence of HDV, natural or artificial variants, analogs, or derivatives thereof, is detected in the sample. In a preferred specific embodiment, the presence of HDV, natural or artificial variants, analogs, or derivatives thereof, is detected in the sample by a quantitative real time polymerase chain reaction (qRT-PCR) using the primers that are constructed based on a partial nucleotide sequence of a fit of all known HDV virus genome sequences.

The primer and probe sequences used in production of an amplicon are specifically tailored and designed to satisfy several different parameters depending on the primer or probe. In general, an amplicon is ideally less than 150 nucleotides, optionally from 75 to 150 nucleotides or any value or range therebetween. In some embodiments, an amplicon should not exceed 150 nucleotides.

A forward primer should be no longer than 19 nucleotides and be used to bind in the region of the HDV in nucleotides 816 to 834 using the numbering of Wang, et al., Nature, 1986; 323: 508-514; corrigendum 328:456 and as found in the NCBI database at GenBank accession number X04451.1. A forward primer for HDV detection should include at least two degenerate nucleotide positions, but may include more than 2 such as 3, 4, or 5.

A reverse primer for detection of HDV optionally has 32 nucleotides or fewer and optionally includes a core region that is suitable to bind HDV in the region of nucleotides 894 to 908 using the numbering of Wang, et al., Nature, 1986; 323: 508-514; corrigendum 328:456 and as found in the NCBI database at GenBank accession number X04451.1, optionally without a mutation or degeneracy. No extensions are permissible beyond the 5′ end of a primer sequence including nucleotides TCCTC at the 5′ end.

A probe for detection of HDV optionally extends between nucleotides 853 and 890 using the numbering of Wang, et al., Nature, 1986; 323: 508-514; corrigendum 328:456 and as found in the NCBI database at GenBank accession number X04451.1. A probe optionally includes at least a core sequence of nucleotides 858-876 and include at least two degenerate nucleotides within those positions.

Exemplary sequences suitable for primers or probes for the detection of HDV are found in Table 1A.

TABLE IA

| Nucleotide | | SEQ ID |
|---|---|---|
| Purpose | Sequence 5′ to 3′ | NO: |
| Forward Primer | TCTCCCTTWGCCATCMGAG | 1 |
| Reverse Primer | TCCTCTTCGGGTCGG | 2 |
| | TCCTCTTCGGGTCGGC | 4 |
| | TCCTCTTCGGGTCGGCATG | 5 |
| Probe | CYCGCGGTCCGWCCTGGGC | 3 |
| | CYCGCGGTCCGWCCTGGCRTCCG | 6 |
| | CCTCCYCGCGGTCCGWCCTGGGC | 7 |
| | CTCCYCGCGGTCCGWCCTGGGC | 8 |
| | TCCYCGCGGTCCGWCCTGGGC | 9 |
| | CCYCGCGGTCCGWCCTGGGC | 10 |

In a non-limiting specific embodiment, a forward primer to be used in a qRT-PCR process includes or consists of 5′-TCTCCCTTWGCCATCMGAG-3′ (SEQ ID NO: 1) where W represents A or T, and M represents A or C. A reverse primer optionally includes or consists of 5′-TCCTCTTCGGGTCGG-3′ (SEQ ID NO: 2). In preferred embodiments, the primers used in the process are SEQ ID NOS: 1 and 2. An agent for detecting HDV nucleic acid sequences is a labeled nucleic acid probe capable of hybridizing to an amplification product produced by a forward and reverse primer pair. In some embodiments, the nucleic acid probe is a nucleic acid molecule comprising or consisting of the nucleic acid sequence of 5′-CYCGCGGTCCGWCCTGGGC-3′ (SEQ ID NO: 3), where Y represents C or T, and W represents A or T. It is appreciated that a nucleotide primer or probe optionally includes one or more locked nucleic acids. LNA denotes a locked nucleic acid. LNAs were first detailed in Koshkin et al., Tetrahedron 54, 3607-3630 (1998).

In some embodiments, a simultaneous or sequential detection of the presence or absence of nucleic acid molecules of HAV, HBV, HCV, HDV, and HEV individually or in any combination, natural or artificial variants, analogs, or derivatives thereof, in a biological sample. Exemplary processes involve obtaining a biological sample from various sources and contacting the sample with a compound or an agent capable of detecting a nucleic acid sequence of HAV, HBV, HCV, HDV, or HEV, natural or artificial variants, analogs, or derivatives thereof, such that the presence of HAV, HBV, HCV, HDV, or HEV, natural or artificial variants, analogs, or derivatives thereof, is detected in the sample. In a preferred specific embodiment, the presence of HAV, HBV, HCV, HDV, or HEV, natural or artificial variants, analogs, or derivatives thereof, is detected in the sample by a quantitative real time polymerase chain reaction (qRT-PCR) optionally using the primers that are constructed based on a partial nucleotide sequence of a fit of all known HDV virus genome sequences.

Exemplary sequences suitable for primers or probes for the detection of the presence or absence of HAV, HBV, HCV, HDV, or HEV or any combination thereof, or all thereof are found in Table 1A and Table 1B.

TABLE IB

| | Nucleotide | | SEQ ID |
|---|---|---|---|
| Virus | Purpose | Sequence 5′ to 3′ | NO: |
| HAV | Forward Primer | GGG TGA AAC CTC TTA GGC TAA TAC | 16 |
| | Reverse Primer | TCC TCC GGC GTT GAA TG | 17 |
| | | CAC CAA TAT CCG CCG CTG TTA CCC TAT | |
| | Probe | CCA | 18 |
| HBV | Forward Primer | TGT CCT GGY TAT CGC TGG AT | 19 |
| | Reverse Primer | AAG AAC CAA YAA GAA GAT GAG | 20 |
| | | TGC GGC GTT TTA TCA TMT “T”CC TCT | |
| | Probe | TCA T | 21 |
| HCV | Forward Primer | CTA GCC GAG TAG YGT TGG GT | 22 |
| | Reverse Primer | CAT GTT GCA CGG TCT ACG AG | 23 |
| | Probe | CTC GCA AGC ACC CTA TCA GGC AGT AC | 24 |
| HEV | Forward Primer | GGT GGT TTC TGG GGT GAC | 25 |
| | Reverse Primer | AGG GGT TGG TTG GAT GAA | 26 |
| | Probe | TGA TTC TCA GCC CTT CGC | 27 |

Optionally, a process is used to detect the presence or absence of HDV and HAV. Optionally, a process is used to detect the presence or absence of HDV and HBV. Optionally, a process is used to detect the presence or absence of HDV and HCV. Optionally, a process is used to detect the presence or absence of HDV and HEV. Optionally, a process is used to detect the presence or absence of HAV and HBV. Optionally, a process is used to detect the presence or absence of HAV and HCV. Optionally, a process is used to detect the presence or absence of HAV and HEV. Optionally, a process is used to detect the presence or absence of HBV and HCV. Optionally, a process is used to detect the presence or absence of HBV and HEV. Optionally, a process is used to detect the presence or absence of HCV and HEV. Optionally, a process is used to detect the presence or absence of three of HAV, HBV, HCV, HDV, or HEV in any combination. Optionally a process is used to detect the presence or absence of four of HAV, HBV, HCV, HDV, or HEV. Optionally a process is used to detect the presence or absence of all of HAV, HBV, HCV, HDV, and HEV.

The processes of the present invention can involve a real-time quantitative PCR assay. In a preferred embodiment, the quantitative PCR used in the present invention is a TaqMan assay (Holland et al., PNAS 88(16):7276 (1991)). It is appreciated that the current invention is amenable to performance on other RT-PCR systems and protocols that use alternative reagents illustratively including, but not limited to Molecular Beacons probes, Scorpion probes, multiple reporters for multiplex PCR, combinations thereof, or other DNA detection systems.

The assays are performed on an instrument designed to perform such assays, for example those available from Applied Biosystems (Foster City, Calif.). In more preferred specific embodiments, the present invention provides a real-time quantitative PCR assay to detect the presence of any species of hepatitis virus selected from HAV, HBV, HCV, HDV, or HEV, natural or artificial variants, analogs, derivatives thereof, or any combination thereof, in a biological sample by subjecting the hepatitis virus nucleic acid from the sample to PCR reactions using specific primers, and detecting the amplified product using a probe.

The term “HXV” as used herein means: HAV; HBV; HCV; HDV; HEV; HDV and HAV; HDV and HBV; HDV and HCV; HDV and HEV; HAV and HBV; HAV and HCV; HAV and HEV; HBV and HCV; HBV and HEV; HCV and HEV; any three of HAV, HBV, HCV, HDV, or HEV in any combination; any four of HAV, HBV, HCV, HDV, or HEV in any combination; or all of HAV, HBV, HCV, HDV, and HEV.

In preferred embodiments, the probe is a TaqMan probe which consists of an oligonucleotide with a 5′-reporter dye and a 3′-quencher dye. In some embodiments, a fluorescent reporter dye, such as FAM dye (illustratively 6-carboxyfluorescein), is covalently linked to the 5′ end of the oligonucleotide probe. Other dyes illustratively include TAMRA, AlexaFluor dyes such as AlexaFluor 495 or 590, Cascade Blue, Marina Blue, Pacific Blue, Oregon Green, Rhodamine, Fluoroscein, TET, HEX, Cy5, Cy3, and Tetramethylrhodaminc. Each of the reporters is quenched by a dye at the 3′ end or other non-fluorescent quencher. Quenching molecules are suitably matched to the fluorescence maximum of the dye. Any suitable fluorescent probe for use in real-time PCR (RT-PCR) detection systems is illustratively operable in the instant invention. Similarly, any quenching molecule for use in RT-PCR systems is illustratively operable. In a preferred embodiment a 6-carboxyfluorescein reporter dye is present at the 5′-end and matched to Black Hole Quencher (BHQI, Biosearch Technologies, Inc., Novato, Calif.). The fluorescence signals from these reactions are captured at the end of extension steps as PCR product is generated over a range of the thermal cycles, thereby allowing the quantitative determination of the viral load in the sample based on an amplification plot.

The HXV virus nucleic acid sequences are optionally amplified before being detected. The term “amplified” defines the process of making multiple copies of the nucleic acid in either RNA or cDNA form from a single or lower copy number of nucleic acid sequence molecule. The amplification of nucleic acid sequences is carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, AmpliTaq Gold DNA Polymerase from Applied Biosystems, other available DNA polymerases, reverse transcriptase (preferably iScript RNase H+ reverse transcriptase), ligase, and other enzymes, including heat-stable enzymes (i.e., those enzymes that perform primer extension after being subjected to temperatures sufficiently elevated to cause denaturation). In a preferred embodiment, the AgPath-ID One Step Kit by Applied Biosystems is used. Suitable enzymes will facilitate combination of the nucleotides in the proper manner to form the primer extension products that are complementary to each mutant nucleotide strand. Generally, the synthesis is initiated at the 3′-end of each primer and proceed in the 5′-direction along the template strand, until synthesis terminates, producing molecules of different lengths. There may be amplification agents, however, that initiate synthesis at the 5′-end and proceed in the other direction, using the same process as described above. In any event, the process of the invention is not to be limited to the embodiments of amplification described herein.

One process of in vitro amplification, which is used according to this invention, may include the polymerase chain reaction (PCR) such as those described in U.S. Pat. Nos. 4,683,202 and 4,683,195. The term “polymerase chain reaction” refers to a process for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. Many polymerase chain processes are known to those of skill in the art and may be used in the process of the invention. For example, RNA is subjected to cycling parameters in a thermocycler optionally as follows: 45° C. for 10 min, 94° C. for 10 min, followed by 45 cycles of 95° C. for 30 sec and 60° C. for 1 min. For another example, RNA is subjected to a polymerase chain in a thermocycler using parameters of denaturing at 94° C. for 10 min followed by 35 cycles of 94° C. for 30 sec, 58° C. for 30 sec and 72° C. for 1 min with a final extension step of 72° C. for 10 min at a denaturing temperature of 95° C. for 30 sec, followed by varying annealing temperatures ranging from 54 to 58° C. for 1 min, an extension step at 70° C. for 1 min, with a final extension step at 70° C. for 5 min.

The primers for use in amplifying the mRNA or genomic RNA of HXV may be prepared using any suitable process, such as conventional phosphotriester and phosphodiester processes or automated embodiments thereof so long as the primers are capable of hybridizing to the nucleic acid sequences of interest. One process for synthesizing oligonucleotides on a modified solid support is described in U.S. Pat. No. 4,458,066. The exact length of primer will depend on many factors, including desired melting temperature, buffer, and nucleotide composition. The primer must prime the synthesis of extension products in the presence of the inducing agent for amplification.

Primers used according to the process of the invention are complementary to each strand of nucleotide sequence to be amplified. The term “complementary” means that the primers must hybridize with their respective strands under conditions that allow the agent for polymerization to function. In other words, the primers that are complementary to the flanking sequences hybridize with the flanking sequences and permit amplification of the nucleotide sequence. Preferably, the 3′ terminus of the primer that is extended is perfectly base paired with the complementary flanking strand. In some embodiments, probes possess nucleotide sequences complementary to one or more strands of the 5′-NCR of HDV. Optionally, one or more primers are complementary to HDV genetic sequences encompassing positions 800-910. Optionally, primers are used in pairs wherein a pair contain the nucleotide sequences of SEQ ID NOs: 1 and 2; 16 and 17; 19 and 20; 22 and 23; 25 and 26; or any combination of the primer pairs. It is appreciated that the complement of SEQ ID NOs: 1, 2, 16, 17, 19, 20, 22, 23, 25, or 26 are similarly suitable for use in the instant invention. It is further appreciated that oligonucleotide sequences that hybridize with SEQ ID NO: 1, 2, 16, 17, 19, 20, 22, 23, 25, or 26 are also similarly suitable. Finally, while it was previously believed that multiple positions are available for hybridization on the HDV genome, the primers used in the present invention, particularly the forward primer are highly restricted both positionally and compositionally so as to provide the proper melting temperature, ability to recognize all known HDV genotypes or all of HAV, HBV, HCV, HDV, and HEV, refrain from formation of secondary structure, and have a specific ability to bind with sufficient hybridization to the genome so as to be useful in the invention.

Those of ordinary skill in the art will know of various amplification processes that can also be utilized to increase the copy number of a target HXV nucleic acid sequence. The nucleic acid sequences detected in the process of the invention are optionally further evaluated, detected, cloned, sequenced, and the like, either in solution or after binding to a solid support, by any process usually applied to the detection of a specific nucleic acid sequence such as another polymerase chain reaction, oligomer restriction (Saiki et al., BioTechnology 3:1008 1012 (1985)), allele-specific oligonucleotide (ASO) probe analysis (Conner et al., PNAS 80: 278 (1983)), oligonucleotide ligation assays (OLAs) (Landegren et al., Science 241:1077 (1988)), RNase Protection Assay and the like. Molecular techniques for DNA analysis have been reviewed (Landegren et al., Science 242:229 237 (1988)). Following DNA amplification, the reaction product may be detected by Southern blot analysis, without using radioactive probes. In such a process, for example, a small sample of DNA containing the nucleic acid sequence obtained from the tissue or subject is amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

The term “labeled” with regard to the probe is intended to encompass direct labeling of the probe by coupling (i.e., physically linking) a detectable substance to the probe, as well as indirect labeling of the probe by reactivity with another reagent that is directly labeled. Examples of indirect labeling include detection of a probe using a fluorescently labeled antibody and end-labeling or centrally labeling of a DNA probe with biotin such that it can be detected with fluorescently labeled streptavidin. The detection method of the invention can be used to detect RNA (particularly mRNA) or genomic nucleic acid in a sample in vitro as well as in vivo. For example, in vitro techniques for detection of nucleic acid include northern hybridizations, in situ hybridizations, RT-PCR, real-time RT-PCR, and DNase protection. Furthermore, in vivo techniques for detection of HXV include introducing into a subject organism a labeled antibody directed against a capsid or polypeptide component or directed against a particular nucleic acid sequence of HXV. For example, the antibody can be labeled with a radioactive marker whose presence and location in the subject organism can be detected by standard imaging techniques, including autoradiography.

The size of the primers used to amplify a portion of the nucleic acid sequence of HXV is at least 5, and often 10, 15, 20, 25, or 30 nucleotides in length. Optionally, a primer is 10-30 nucleotides in length or any value or range therebetween. Preferably, the GC ratio should be above 30%, 35%, 40%, 45%, 50%, 55%, or 60% so as to prevent hair-pin structure on the primer. Furthermore, the amplicon should be sufficiently long enough to be detected by standard molecular biology methodologies. Although it is not desired in the invention, techniques for modifying the Tₘ of either primer are operable herein. An illustrative forward primer contains LNA-dA and LNA-dT (Glen Research Corporation) so as to match Tₘ with a corresponding alternate primer.

An inventive process uses a polymerization reaction which employs a nucleic acid polymerizing enzyme, illustratively a DNA polymerase, RNA polymerase, reverse transcriptase, or mixtures thereof. It is further appreciated that accessory proteins or molecules are present to form the replication machinery. In a preferred embodiment the polymerizing enzyme is a thermostable polymerase or thermodegradable polymerase. Use of thermostable polymerases is well known in the art such as Taq polymerase available from Invitrogen Corporation. Thermostable polymerases allow a polymerization reaction to be initiated or shut down by changing the temperature other condition in the reaction mixture without destroying activity of the polymerase.

Accuracy of the base pairing in the preferred embodiment of DNA sequencing is provided by the specificity of the enzyme. Error rates for Taq polymerase tend to be false base incorporation of 10-s or less. (Johnson, Annual Reviews of Blochemistry, 1993: 62:685-713; Kunkel, Journal of Biological Chemistry, 1992; 267:18251-18254). Specific examples of thermostable polymerases illustratively include those isolated from Thermus aquaticus, Thermus thermophilus, Pyrococcus woesei, Pyrococcus furiosus, Thermococcus litoralis and Thermotoga maritima. Thermodegradable polymerases illustratively include E. coli DNA polymerase, the Klenow fragment of E. coli DNA polymerase, T4 DNA polymerase, T7 DNA polymerase and other examples known in the art. It is recognized in the art that other polymerizing enzymes are similarly suitable illustratively including E. coli, T7, T3, SP6 RNA polymerases and AMV, M-MLV, and HIV reverse transcriptases.

The polymerases are optionally bound to the primer. When the HXV is a single-stranded RNA molecule due to heat denaturing the polymerase is bound at the primed end of the single-stranded nucleic acid at an origin of replication. A binding site for a suitable polymerase is optionally created by an accessory protein or by any primed single-stranded nucleic acid.

Preferably, PCR amplification products are generated using complementary forward and reverse oligonucleotide primers. In a non-limiting example, HDV genetic sequences or fragments thereof are amplified by the primer pair SEQ ID NOS: 1 and 2 that amplify a conserved sequence in the HDV 5′-NCR encompassing nucleotides 816-908. The resulting amplification product is processed and prepared for detection by processes known in the art. It is appreciated that the complements of SEQ ID NOS: 1 and 2 are similarly suitable for use in the instant invention. It is further appreciated that oligonucleotide sequences that hybridize with SEQ ID NO: 1 or 2 are also similarly suitable.

Optionally, multiple amplification products are simultaneously produced in a PCR reaction that is then available for simultaneous detection and quantification. Thus, multiple detection signals are inherently produced or emitted that are separately and uniquely detected in one or more detection systems. It is appreciated that multiple detection signals are optionally produced in parallel. Preferably, a single biological sample is subjected to analysis for the simultaneous or sequential detection of HXV genetic sequences. It is appreciated that three or more independent or overlapping sequences are simultaneously or sequentially measured in the instant inventive process. Oligonucleotide matched primers (illustratively SEQ ID NOS: 1 and 2) are simultaneously or sequentially added and the biological sample is subjected to proper thermocycling reaction parameters. For detection by mass spectrometry a single sample of the amplification products from each gene are simultaneously analyzed allowing for rapid and accurate determination of the presence of HXV. Optionally, analysis by qRT-PCR is employed capitalizing on multiple probes with unique fluorescent signatures. Thus, each genome is detected without interference by other amplification products. This, multi-target approach increases confidence in quantification and provides for additional internal control.

In a specific embodiment, the processes further involve obtaining a control sample from a control subject, contacting the control sample with a compound or agent capable of detecting the presence of HXV nucleic acid in the sample, and comparing the presence of mRNA or genomic RNA in the control sample with the presence of mRNA or genomic RNA in the test sample.

In a further embodiment detection of PCR products is achieved by mass spectrometry. Mass spectrometry has several advantages over RT-PCR or qRT-PCR systems in that it similarly can be used to simultaneously detect the presence of HXV and decipher mutations in target nucleic acid sequences allowing identification and monitoring of emerging strains. Further, mass spectrometers are prevalent in the clinical laboratory. Similar to fluorescence based detection systems mass spectrometry is capable of simultaneously detecting multiple amplification products for a multiplexed and controlled approach to accurately quantifying components of biological or environmental samples.

Multiple mass spectrometry platforms are suitable for use in the instant invention illustratively including matrix assisted laser desorption ionization time of flight mass spectrometry (MALDI), electrospray mass spectrometry, electrospray ionization-Fourier transform ion cyclotron resonance mass spectrometry (ESI-FTICR), multi-stage mass spectrometry fragmentation analysis (MS/MS), mass spectrometry coupled with liquid chromatography such as high performance liquid chromatography mass spectrometry (HPLC) and ultra performance liquid chromatography isotope dilution tandem mass spectrometry (UPLC-ID/MS/MS), variations thereof, or combinations thereof.

It is appreciated that numerous other detection processes are similarly suitable for measuring an amplification product by detecting a detection signal. Illustrative examples include, but are not limited to, liquid chromatography, mass spectrometry, liquid chromatography/mass spectrometry, static fluorescence, dynamic fluorescence, high performance liquid chromatography, ultra-high performance liquid chromatography, enzyme-linked immunoadsorbent assay, qRT-PCR, gel electrophoresis, fluorescence resonance energy transfer, nucleotide sequencing, enzyme-linked immunoadsorbent assay, affinity chromatography, chromatography, immunoenzymatic methods (Ortiz, A and Ritter, E, Nucleic Acids Res., 1996; 24:3280-3281), streptavidin-conjugated enzymes, DNA branch migration (Lishanski, A, et al., Nucleic Acids Res., 2000; 28(9):e42), enzyme digestion (U.S. Pat. No. 5,580,730), colorimetric methods (Lee, K., Biotechnology Letters, 2003; 25:1739-1742), or combinations thereof.

In some embodiments, TaqMan Array Cards (TAC, Life Technologies, Grand Island, N.Y.), formerly known as TaqMan Low Density Array (TLDA) are used in an assay for rapid and simultaneous detection of HAV RNA, HBV DNA, HCV RNA, HDV RNA and HEV RNA. TAC is a set of 384 wells of 1 μl capacity each, interconnecting 48 individual wells on a plastic array card to one central port which can accommodate one clinical specimen. Each card contains eight identical ports, providing a platform to test six to eight clinical specimens simultaneously by real-time PCR, depending on how many control specimens are run.

TAC-ready assays provide a potential for building modular diagnostic platforms for acute and chronic viral hepatitis, jaundice syndromic surveillance, blood-borne pathogens screening of blood or solid organ donors and recipients, and testing at-risk populations, including intravenous drug users and hemodialysis patients.

The invention also encompasses kits for detecting the presence of HXV viral nucleic acids in a test sample. The kit, for example, includes a labeled compound or agent capable of detecting a nucleic acid molecule in a test sample and, in certain embodiments, for determining the titer in the sample.

For oligonucleotide-based kits, the kit includes for example: (1) an oligonucleotide, e.g., a detectably labeled probe, which hybridizes to a nucleic acid sequence of the HXV virus and/or (2) a pair of primers (one forward and one reverse) useful for amplifying a nucleic acid molecule containing the HXV viral sequence. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which is assayed and compared to the test sample contained. Each component of the kit is usually enclosed within an individual container and all of the various containers are usually enclosed within a single package along with instructions for use. A kit optionally includes a primer including a nucleotide sequence of SEQ ID NO: 1, 2, 16, 17, 19, 20, 22, 23, 25, or 26, or combinations of the primers. A kit optionally includes a probe of SEQ ID NO: 3, 18, 21, 24, or 27, or combinations of the probes.

The inventive processes, kits and compositions are amenable to use for diagnosis of HXV infection in a subject, insects, and any inclusive other organism capable of infection or transfection by or with HXV.

To increase confidence and to serve as an internal or external control, a purified and titered HXV solution is optionally used as a biological sample. By amplification of a single sample with known quantities of HXV or of a set of samples representing a titration of HXV, the level of HXV in the unknown biological sample is determined. Preferably, the purified and titered HXV solution is analyzed in parallel with the unknown biological sample to reduce inter assay error or to serve as a standard curve for quantitation of unknown HXV in the biological sample. Using purified and titered HXV solution provides for a similar complete genetic base RNA strand for amplification.

In another embodiment, a subgenomic fragment is cloned into a plasmid for amplification, purification, and use as a quantitative comparator or nucleic acid calibrator. In a non-limiting example, a RNA subgenomic fragment of HDV is optionally amplified from a positive plasma sample using primers bracketing the qRT-PCR target regions in the 5′-NCR of HDV. It is appreciated that other sequences are similarly suitable for use as a quantitative control. The known concentration of the subgenomic fragment is used to create a standard curve for quantitative determinations and to access amplification efficiency.

Also provided is a kit for detecting HXV infection that contains reagents for the amplification, or direct detection of HXV or portions thereof. An exemplary kit illustratively includes a forward and reverse primer pair, and a probe. An exemplary embodiment, the forward and reverse primers have the oligonucleotide sequence SEQ ID NOS: 1 and 2 and a nondegenerate probe of the sequence SEQ ID NO: 3. In an exemplary embodiment, a kit includes one or more primers or probes as listed in Table 1A and 1B. It is appreciated that a diagnostic kit may optionally contain primers and probes that are the complements of SEQ ID NOS: 1-3 or that hybridize with oligonucleotides SEQ ID NOS: 1-3, or a complements or hybridize with any other probe or primer from Table 1A and 1B. It is further appreciated that a diagnostic kit optionally includes ancillary reagents such as buffers, solvents, thermostable polymerases, nucleotides, and other reagents necessary and recognized in the art for amplification and detection of HXV in a biological sample.

The invention provides a host cell containing a nucleic acid sequences according to the invention as an alternative to synthetic primer sequence generation. Plasmids containing the polymerase components of the HXV virus are generated in prokaryotic cells for the expression of the components in relevant cell types (bacteria, insect cells, eukaryotic cells). Preferably, the cell line is a primate cell line. These cell lines may be cultured and maintained using known cell culture techniques such as described in Celis, Julio, ed., 1994. Cell Biology Laboratory Handbook, Academic Press, NY. Various culturing conditions for these cells, including media formulations with regard to specific nutrients, oxygen, tension, carbon dioxide and reduced serum levels, can be selected and optimized by one of skill in the art.

The preferred cell line of the present invention is a eukaryotic cell line, preferably an insect cell line, such as Sf9 per, transiently or stably capable of expressing one or more full-length or partial HXV proteins. Such cells can be made by transfection (proteins or nucleic acid vectors), infection (viral vectors) or transduction (viral vectors). The cell lines for use in the present invention are cloned using known cell culture techniques familiar to one skilled in the art. The cells are cultured and expanded from a single cell using commercially available culture media under known conditions suitable for propagating cells.

A host cell is a cell derived from a mammal, insect, yeast, bacteria, or any other single or multicellular organism recognized in the art. Host cells are optionally primary cells or immortalized derivative cells. Immortalized cells are those which can be maintained in-vitro for several replication passages.

In an exemplary embodiment, an HXV antigen such as an amino acid sequence representative of a capsid protein is used as a control for a PCR based assay for the detection and measurement of the presence of HXV in a biological sample. The process of detecting HXV antibodies in a biological sample is optionally performed in parallel with the same or control biological samples that are used to detect HXV genetic sequences.

A kit for detection of HXV infection in a patient optionally contains reagents for PCR based detection of HXV genetic sequences, either structural or non-structural, and optionally for detection of antibodies directed to structural HXV proteins. The components of the kits are any of the reagents described above or other necessary and non-necessary reagents known in the art for solubilization, detection, washing, storage, or other need for in a diagnostic assay kit.

Overall, the provided inventive processes and compositions can be used as a quantitative one-step RT-PCR assay or other assay for HXV RNA detection and quantitation. In some embodiments, the processes use a simple one-step qRT-PCR set or reactions that can be run on various real-time PCR instruments from as little as 200 μl of serum. While most published HDV RNA assays target the delta antigen sequence, the inventive assay for HDV targets the adjacent region located upstream from the delta antigen, in view of its higher level of conservation among all eight delta genotypes. Another advantage of our assay is that the inventive positive control transcript controls for every step of the qRT-PCR reaction, including the reverse transcription step. We have also described the preparation of HXV transcripts for use as a positive control. Both the qRT-PCR and transcript preparation are useful for detection and quantitation of HXV RNA for diagnostic and surveillance purposes.

The present invention is further illustrated with respect to the following non-limiting examples. The following examples are for illustrative purposes only and are not a limitation on the practice or scope of the invention.

### EXAMPLES

### Example 1

### HDV Primer and Probe Design

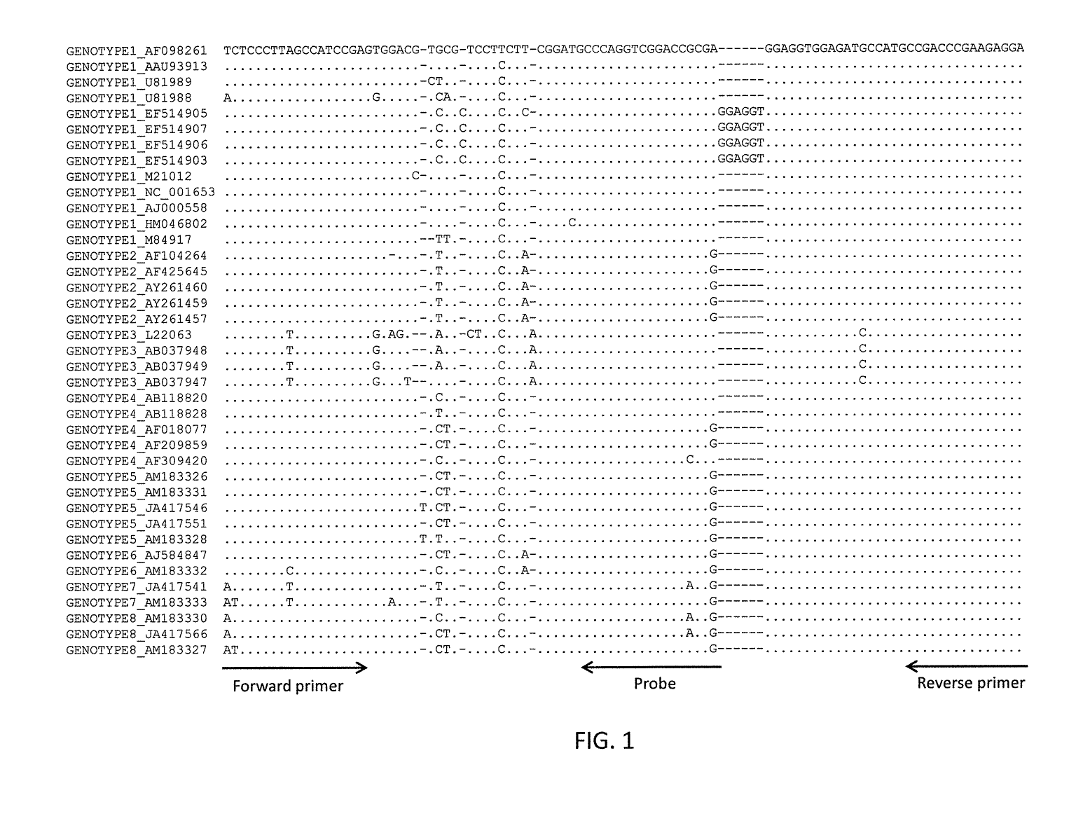

Full-length HDV genome sequences downloaded from GenBank (Benson et al. 2010) were aligned using MEGA 5 software (Tamura et al., 2011). Gaps were allowed in the alignment to account for differences between genotypes. The sequence alignment was used to find conserved regions of the genome to identify potential regions for HDV RNA detection (FIG. 1). The alignment consisted of full HDV cDNA sequences which included the ORF for the L-HDAg and full-length sequences.

Using the sequence alignment, multiple primers and probes were tested with primers PAN-HDVF and PAN-HDVR and probe PAN-HDVP demonstrating significantly better analytical performance (Table 1). These primers amplify a sequence located upstream of the L-HDAg ORF (FIG. 1). All primers and probes were analyzed for melting temperature, primer-dimer formation, self-annealing, and secondary structure formation using the Oligoanalyzer software. All primer and probe sequences were analyzed by BLAST to verify sequence uniqueness of the chosen regions.

### Example 2

### Obtaining Viral Strains and Clinical Specimens

For HDV, the evaluation panel included 132 HBsAg/anti-HDV positive sera previously referred to our laboratory that had been stored at −70° C. These sera originated from the United States, Moldova, Romania and Venezuela. Qualitative confirmation of the HBsAg-positive status was determined using the VITROS ECi Immunodiagnostic System (Ortho-Clinical Diagnostics, Inc., Rochester, N.Y.). Total anti-HDV was detected using the EIA-AB DELTAK-2 Kit (DiaSorin, Stillwater, Minn.): positive samples were tested for HDV RNA in two independent runs by the qRT-PCR assay.

For a multiplex assay for the detection of HAV, HBV, HCV, HDV, and HEV, the following commercially available standards were used for development and optimization of multiplex PCR assay for detection of all five hepatitis viruses: HAV RNA Working Reagent for Nucleic Acid Amplification, code 01/488 (NIBSC, South Mimms, United Kingdom); AcroMetrix HBV Panel, reference 950150 (Life Technologies, Grand Island, N.Y.); AcroMetrix HCV Panel, reference 942011 (Life Technologies, Grand Island, N.Y.); and WHO International Standard for Hepatitis E Virus RNA Nucleic Acid Amplification Techniques (NAT)-Based Assays, code 6329/10 (Paul-Ehrlich-Institut, Langen, Germany). Furthermore, the following genotyping panels were used: HBV DNA Genotype Performance Panel PHD201(M) and HCV RNA Genotype Performance Panel PHW202 (SeraCare Life Sciences, Milford, Mass.). In addition, serum specimens previously tested and confirmed positive for HAV RNA (n=46), HBV DNA (n=39). HCV RNA (n=32), HDV RNA (n=28) and HEV RNA (n=14) were used to evaluate the multiplex assay. Control samples negative for HAV RNA (n=41), HBV DNA (n=32), HCV RNA (n=36), HDV RNA (n=30), and HEV RNA (n=31) were included to test the specificity of the assay.

### Example 3

### Extraction of Viral Nucleic Acids

Total nucleic acid was extracted from 200 μL of each serum specimen using the MagNA Pure LC 2.0 Instrument and the MagNA Pure Total Nucleic Acid Isolation Kit (Roche Diagnostics, Indianapolis, Ind.). All extractions were performed using 200 μl of serum or plasma according to the manufacturer's protocols; TNAs were eluted in 50 μl of the Elution buffer. TNAs were aliquoted and stored at −80° C. until use.

### Example 4

### One-Step qRT-PCR

qRT-PCR reactions were performed on the ABI 7500 real-time PCR instrument using the AgPath-ID One-Step Kit (Applied Biosystems, Foster City, Calif.) following the manufacturer-recommended protocols. All reactions were performed in 96-well plates in 25 μl reaction volume, containing IX RT-PCR enzyme mix, lx RT-PCR buffer, 600 nM each primer and 200 nM probe, and 5 μl of TNA extract. The following cycling conditions were used: 45° C. for 10 min, 94° C. for 10 min, followed by 45 cycles of 95° C. for 30 sec and 60° C. for 1 min.

To satisfy the necessary parameters for fluorescence intensity level, efficiency, and specificity of the HDV qRT-PCR assay, various concentrations as well as ratios of primers and probes were tested. As an HDV RNA standard for quantification of HDV RNA by PCR is not available, we sourced, for optimization purposes, an anti-HDV-positive human serum specimen from which 10-fold serial dilutions were made. Among the few primers and probe sequences that were suitable for use in the assay, the primers of SEQ ID Nos: 1 and 2 and the probe of SEQ ID NO:3 were chosen for particular additional exemplary use. Utilizing 600 nM of each primer and 200 nM of probe in the qRT-PCR reaction was determined to achieve the best detection sensitivity: HDV RNA could be detected down to the 10⁻⁵ dilution of the serum. Specificity was determined using 48 anti-HDV-negative human sera (SeraCare, Milford, Mass.); HDV RNA was not detected in any of the specimens. When applied to test the HBV DNA Genotype Performance Panel PHD201(M) (SeraCare), no positive reactions were observed, suggesting no interference from the presence of HBV DNA.

### Example 5

### Detection and Quantitation of HDV RNA in Clinical Samples

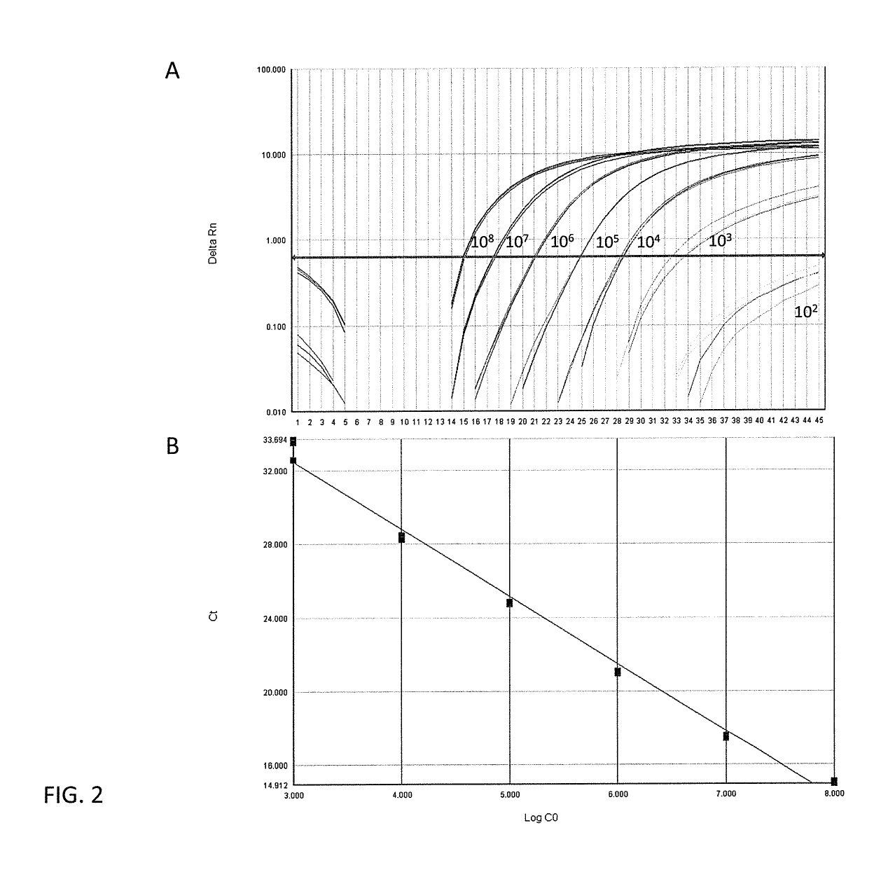

Of the 132 specimens of Example 1 that tested positive for anti-HDV, 41 (31.06%) were positive for HDV RNA by the TaqMan qRT-PCR assay. We also determined the HDV RNA viral titers in all HDV RNA positive samples. To generate a standard curve, serial dilutions of the transcript preparation were made, ranging from 10⁸ to 10¹ transcript copies per reaction. The assay exhibited a 6-log linear range with a lower detection limit of 7.5×10² copies/ml and an upper detection limit of 7.5×10⁸ copies/ml. The slope of −3.65 suggested the qRT-PCR efficiency of over 90% (FIG. 2). A 10-fold dilution series of the positive control transcript was used to determine the HDV RNA titers of the 41 serum specimens that tested positive for HDV RNA. The mean viral titer of the 41 HDV RNA positive sera was 1.3×10⁸ copies/ml (range 45.2-1.79×10⁹).

All HDV RNA positive samples were further characterized for genotype by sequencing the HDAg region using the nested primers listed in Table 2 followed by sequencing.

TABLE 2

| | | SEQ ID |
|---|---|---|
| Nucleotide Purpose | Sequence 5 to 3′ | NO: |
| Nested PCT External | AGAAAGCAACGGGGCTAG | 11 |
| Fwd Primer | | |
| Nested PCT External | TTCCTCTTCGGGTCGGCA | 12 |
| Rev Primer | | |
| Nested PCT External | TCTTTCCTCTTCGGGTCG | 13 |
| Rev Primer (Alt) | | |
| Nested PCT Internal | TGCCGAGTGAGGCTTATC | 14 |
| Fwd Primer | | |
| Nested PCT Internal | GAGGAGGCTGGGACCATG | 15 |
| Rev Primer | | |

The 257-base pair amplicon used for genotyping is located in the L-HDAg ORF. The first step of the nested PCR reaction was performed using the AgPath-ID buffer as described above and the DESP and DEAP primers. If no amplicon was obtained, the DEAP-alt reverse primer was used in an otherwise identical reaction. The nested reaction was performed using the DISP and DIAP primers and the PerfeCTa™ SYBR® Green SuperMix, Low ROX (Quanta Biosciences, Gaithersburg, Md.). The cycling conditions for the second PCR reaction, using nested primers DISP and DIAP, were as follows: 94° C. for 10 min followed by 35 cycles of 94° C. for 30 sec, 58° C. for 30 sec and 72° C. for 1 min with a final extension step of 72° C. for 10 min. The amplicons from the second nested PCR reaction were run on a 2% agarose gel to confirm the amplification status and the size of the amplicon.

The nested amplicons were sequenced directly from the second nested PCR reaction using each internal nested primer in a separate reaction for bidirectional sequencing, the BigDye Terminator v3.1 Cycle Sequencing Kit (Applied Biosystems, Foster City, Calif.) according to the manufacturer's instructions, and the 3130×1 Genetic Analyzer (Applied Biosystems, Foster City, Calif.).

Some of the HDV RNA-positive samples yielded HDV-specific sequences and could be genotyped; 15 of 41 belonged to genotype 1 and four belonged to genotype 3. The genotype 1 samples originated from the United States (n=4), Romania (n=1), and Moldova (n=10). The genotype 3 samples originated from Venezuela.

### Example 6

### Detection of HDV Amplicons Via Mass Spectroscopy

Detection of amplification products obtained as in Example 4 will be performed essentially as described by Blyn, L. et al. J. Clin. Microbiol. 2008; 46(2):644-651. Following amplification each PCR mixture will be desalted and purified using a weak anion-exchange protocol based on the method of Jiang and Hofstadler (Jiang, Y., and S. A. Hofstadler. Anal. Biochem. 2003; 316:50-57). ESI-TOF will be used to obtain accurate-mass (±1 ppm), high-resolution (M/ΔM, >10,000 full width half maximum) mass spectra. For each sample, approximately 1.5 μl of analyte solution will be consumed during the spectral acquisition. Raw mass spectra will be post-calibrated with an internal mass standard and deconvolved to average molecular masses. Quantitative results are obtained by comparing the peak heights with an internal PCR calibration standard present in every PCR well at 300 molecules unless otherwise indicated.

### Example 7

### TAC-Chemistry Compatible Individual Real-Time PCR Assay Protocols

A quantitative RT-PCR assay was desiged for detection of HAV RNA, HBV DNA, HCV RNA, HDV RNA and HEV RNA, hereafter referred to as HABCDE-NAs, using TAC compatible chemistry and identical cycling conditions. In the initial evaluation, primers and probes routinely used in our laboratory for nucleic acid testing (NAT) of HAV, HBV, HCV and HEV (Garson et al., Journal of virological methods 186:157-160; Jothikumar N, et al. Journal of virological methods 131:65-71) were transferred into the TAC-compatible buffer system and PCR cycling conditions, and compared to the original assay for their performance characteristics. TAC-chemistry compatible assay reagents for detection of HDV RNA are presented in Table 1.

Real-time RT-PCR reactions were performed on the ABI 7500 real-time PCR instrument using the AgPath-ID One-Step kit (Life Technologies) following the manufacturer-recommended protocols. All reactions were performed in 96-well plates in 25 μl reaction volume, containing 1×RT-PCR enzyme mix, 1×RT-PCR buffer, variable concentrations of each primer and probe (Table 1), and 5 μl of TNA extract. The following cycling conditions were used: 45° C. for 10 min, 94° C. for 10 min, followed by 45 cycles of 95° C. for 30 sec and 60° C. for 1 min.

Primers and probes used for amplification and detection of HABCDE-NAs in this study are shown in Tables 1A and 1B. All the primers and probes were analyzed by BLAST to ensure specificity and their Tm values were theoretically matched with the ideal 60° C. for primers and 70° C. for probes. Since identical chemistry and PCR cycling conditions is a requirement for all assays to be concurrently run on TAC, primer and probe concentrations were carefully identified to provide the combination with highest specificity and best limit of detection.

Once suitable conditions were established in the individual real-time RT-PCR assays, the HABCDE-NA assays were run on TAC in triplicate wells. In addition, two experimental controls were used in duplicate: 1) an internal positive control—IPC2, for checking the card performance and 2) ribo-nuclear protein 3 (RNP3) control, for nucleic acids extraction. Each card has eight different ports, which were utilized to run six specimens in addition to positive and negative controls. A synthetic positive control for HABCDE-NAs, described below, was used as a positive control for all assays in all runs.

A positive control transcript was developed to encompass detection of HABCDE-NAs as described previously. Briefly, a synthetic gene was designed containing the forward primer sequence, the probe sequence, and the reverse complement of the reverse primer sequence for HAV RNA, HBV DNA. HCV RNA, HDV RNA, HEV RNA and RNP3 targets respectively, followed by the reverse complement of the SP6 promoter sequence. This compound sequence was synthesized and cloned into pIDTSmart Amp vector (IDT, Coralville, Iowa). For use as a control, transcripts were generated from the plasmid using the SP6 MEGAscript kit according to the manufacturer's instructions (Life Technologies). The transcripts were purified using the MEGAclear kit according to the manufacturer's instructions (Life Technologies) and contained 2.53×10¹⁵ copies/ml based on the NanoDrop spectrophotometer (Thermo Scientific, Waltham, Mass.) and the Endmemo software (http://endmemo.com/bioidnacopynum.php). The transcript preparation was serially diluted from the 10⁸ copies/ml dilution in 1×TE buffer containing 50 ng/ml of carrier RNA, aliquoted and stored at −70° C. until use.

TAC assays were run on the ViiA7 instrument (Life Technologies) using the AgPath-ID One-Step Kit (Life Technologies). All reactions contained 1×RT-PCR enzyme mix, 1×RT-PCR buffer, and 40 μl of TNA extract. Each sample was set up in 100 μl final reaction and loaded entirely into the TAC port. The universal workflow for setting up PCR reactions, as described above, was followed with set up of TAC reactions. Upon loading, the cards were centrifuged twice at 336×g for 1 min each time, sealed and loaded into the thermal cycler. The cycling conditions described above were used to run the cards.

The reproducibility of the cards was determined based on the average cycle threshold (Ct) values for 31 cards, which included 93 data points for HAV, HBV, HCV, HDV, and HEV NAT assays each, and 62 data points for two controls, RNP3 and IPC2 assays each. The limit of detection of hepatitis TAC assay was determined using serial dilutions of standards for HAV, HBV, HCV, and HEV and the quantified transcript for HDV. To check if any of the viral genomes would interfere or compromise the detection of other viral genomes in a multiplex assay, we created a set of mixed samples each spiked with one to five hepatitis virus combinations as listed: A, B, C, D, E, AB, AC, AD, AE, BC, BD, BE, CD, CE, DE, ABC, ABD, ABE, ACD, ACE, ADE, BCD, BCE, CDE, ABCD, ABCE, ABDE, ACDE, BCDE, and ABCDE. Finally, the assays were evaluated on 329 clinical specimens.

The TAC-compatible HAV RNA PCR assay exhibited a slope of −3.36, and a linear range of 5 logs, with the limit of detection of 125 ge/ml. The new HAV RNA assay was further evaluated using 17 HAV RNA positive and 33 negative human sera, previously tested by a nested RT-PCR (Nianan, et al., Clinical microbiology reviews 19:63-79). Comparing TAC-compatible HAV qRT-PCR to the HAV nested PCR (Id.), clinical sensitivity of the HAV qRT-PCR assay was determined to be 100% and the specificity was 88% [95% CI: 77-99].

The individual 25 μl assay using primers and probes from Tables 1A and B, for detection of HBV DNA (Using SEQ ID Nos: 1, 2, and 3) exhibited a slope of −3.229, and a linear range of 7 logs, with the limit of detection of 100 IU/ml; the individual 25 μl assay for detection of HCV RNA exhibited a slope of −3.327, and a linear range of 7 logs, with the limit of detection of 100 IU/ml; the individual 25 μl assay for detection of HDV RNA exhibited a slope of −3.65, and a linear range of 6 logs, with the limit of detection of 1000 ge/ml; finally, the individual 25 μl assay for detection of HEV RNA exhibited a slope of −3.238, and a linear range of 7 logs, with the limit of detection of 250 IU/ml.

For a multiplex assay for simultancious detection of HABCDE-NAs, the parameters of precision of hepatitis TAC as determined by the positive control transcript, shown in Table 3, indicated the amplification reproducibility of 100%. Furthermore, the Ct values of HABCDE-NAs assays were highly reproducible with tight 95% confidence intervals and low standard deviation.

TABLE 3
TAC precision measurements based on the positive
control transcript data generated from 31 cards.

| | | | | | Stan- |
|---|---|---|---|---|---|
| | | Mini- | Maxi- | | dard |
| | Assay | mum | mum | | Devi- |
| Assay | (n*) | Ct | Ct | Average [95% CI] | ation |
| HAV | 93 | 17.901 | 21.744 | 19.747 [19.589-19.896] | 0.731 |
| HBV | 93 | 19.441 | 23.723 | 20.845 [20.678-21.013] | 0.824 |
| HCV | 93 | 17.116 | 20.142 | 19.398 [19.247-19.549] | 0.745 |
| HDV | 93 | 19.316 | 21.940 | 20.689 [20.565-20.812] | 0.608 |
| HEV | 93 | 17.285 | 22.366 | 20.039 [19.867-20.212] | 0.849 |
| RNP3 | 62 | 20.988 | 23.585 | 22.533 [22.382-22.684] | 0.606 |
| IPC2 | 62 | 18.347 | 21.413 | 19.644 [19.479-19.809] | 0.661 |

*If the assay was present on the card in triplicate, 93 data points were used in these calculations. If the assay was present in duplicate on the cards, 62 data points were used in these calculations.

The lower limit of detection for HABCDE-NAs assays was compared between TAC and its corresponding individual assays with results illustrated in Table 4.

TABLE 4
Limit of detection of TAC hepatitis assays in
comparison to the individual 25 μl assays.

| Assay | TAC | | Wet Chemistry | | Source of Material |
|---|---|---|---|---|---|
| HAV | 250 | ge/ml | 125 | ge/ml | WHO standard |
| HBV | 500 | IU/ml | 100 | IU/ml | AcroMetrix standard |
| HCV | 500 | IU/ml | 100 | IU/ml | AcroMetrix standard |
| HDV | 1000 | ge/ml | 1000 | ge/ml | Transcript (in-house) |
| HEV | 2,500 | IU/ml | 250 | IU/ml | WHO standard |

*ge = genome equivalents
**IU = international units

The HAV TAC assay was half a log₁₀ less sensitive than its individual assay equivalent; the HBV and HCV TAC assays were one fifth of a log₁₀ less sensitive than their individual assay equivalents; the HDV TAC assay was as sensitive as its individual assay equivalent; and the HEV TAC assay was 1 log₁₀ less sensitive than its individual assay equivalent. The overall limit of detection was established at 250 ge/ml for HAV TAC, 500 IU/ml for HBV TAC, 500 IU/ml for HCV TAC, 1000 ge/ml for HDV TAC, and 2500 IU/ml for HEV TAC.

Amplification and detection of individual viral genomes was not affected by the presence of nucleic acids of any other hepatitis viruses as shown in Table 5.

TABLE 5
Amplification of individual viruses by TAC assay in mixtures of multiple hepatitis viruses.

| Number of | | | | | | | | | | | | | | | |
|---|---|---|---|---|---|---|---|---|---|---|---|---|---|---|---|
| viruses | HAV | | | HBV | | | HCV | | | HDV | | | HEV | | |
| in the | | Ave | | | Ave | | | Ave | | | Ave | | | Ave | |
| mix | N | Ct | SD | N | Ct | SD | N | Ct | SD | N | Ct | SD | N | Ct | SD |
| 1 | 3 | 30.158 | 0.326 | 3 | 22.737 | 0.014 | 3 | 33.117 | 0.37 | 3 | 19.291 | 0.180 | 3 | 29.064 | 0.364 |
| 2 | 12 | 27.568 | 0.392 | 12 | 21.702 | 0.145 | 12 | 31.609 | 0.875 | 12 | 19.313 | 0.125 | 12 | 30.191 | 0.979 |
| 3 | 18 | 26.749 | 0.233 | 15 | 21.823 | 0.197 | 18 | 32.928 | 0.844 | 15 | 20.121 | 0.606 | 15 | 29.106 | 1.180 |
| 4 | 12 | 27.987 | 0.447 | 12 | 22.464 | 0.347 | 11* | 33.304 | 1.414 | 12 | 20.309 | 0.356 | 12 | 28.382 | 0.446 |
| 5 | 3 | 27.087 | 0.298 | 3 | 22.212 | 0.348 | 3 | 34.165 | 0.182 | 3 | 20.133 | 0.225 | 3 | 27.947 | 0.532 |

** N = number of RT-PCR reactions, Ave Ct = average crossing threshold value, SD = standard deviation

The range of Ct values obtained for amplification of HABCDE-NAs was within the equivalent of one log₁₀, regardless of the presence of one, two, three, four or five hepatitis viruses in the reaction, thus ruling out any interference of viral genomes of more than one hepatitis virus in the same sample.

Based on the testing of 329 human serum specimens, specificity and sensitivity were determined for hepatitis TAC assays with results illustrated in Table 6.

TABLE 6
Sensitivity and specificity of various TAC assays.

| | Concordant | Discordant | Discordant | Concordant | | | | | Overall |
|---|---|---|---|---|---|---|---|---|---|
| Target | Positives | Positives | Negatives | Positives | Sensitivity | 95% CI | Specificity | 95% CI | Concordance |
| HAV | 43 | 3 | 3 | 38 | 94 | 86-100 | 93 | 85-100 | 93 |
| HBV | 36 | 3 | 0 | 32 | 92 | 84-100 | 100 | 100-100 | 96 |
| HCV | 32 | 0 | 0 | 36 | 100 | 100-100 | 100 | 100-100 | 100 |
| HDV | 28 | 0 | 0 | 30 | 100 | 100-100 | 100 | 100-100 | 100 |
| HEV | 14 | 0 | 0 | 31 | 100 | 100-100 | 100 | 100-100 | 100 |
| Total | 153 | 6 | 3 | 167 | 96 | 93-99 | 98 | 96-100 | 97 |

Using the nested PCR assay as a standard, the HAV TAC exhibited 94% [95% CI: 86-100] sensitivity and 93% [95% CI: 85-100] specificity. Compared to an in-house NAT test for HBV DNA, the HBV TAC exhibited 92% [95% CI: 84-100] sensitivity and 100% specificity. The HCV TAC assay was compared to in vitro diagnostic COBAS platform for HCV RNA detection as the standard. The clinical sensitivity and specificity were 100%/o. For the HDV TAC assay, the individual 25 μl assay identical to the TAC assay was used as the standard, since this is the assay developed specifically for HDV RNA as described above. Both clinical specificity and clinical sensitivity for HDV TAC were 100%. Finally, the HEV TAC assay was compared to the HEV qRT-PCR assay used in our laboratory for detection of HEV RNA as the standard. This assay shares identical primers and probes with the HEV TAC assay, but they differ in chemistry and PCR running conditions. According to this comparison, the HEV TAC assay exhibited 100% specificity and sensitivity. Overall, considering the two to ten-fold drop in the limit of detection, the sensitivity of TAC assays was reasonably high, ranging from 92% (HBV) to 100% (HCV, HDV and HEV). The specificity of the TAC assays was high, ranging from 93% (HAV) to 100% (HBV, ICV, HDV, and HEV) (Table 6).

Patent applications and publications mentioned in the specification are indicative of the levels of those skilled in the art to which the invention pertains. The foregoing description is illustrative of particular embodiments of the invention, but is not meant to be a limitation upon the practice thereof. The following claims, including all equivalents thereof, are intended to define the scope of the invention.

The invention is hereby described with relation to the following references and those otherwise identified in the instant specification. Each reference is incorporated herein by reference as if each were laid out explicitly in its entirety in the instant specification including both text and figures. Each reference is incorporated for the individual point referred to in the specification as well as for all information contained therein and not explicitly identified in the specification. All references are representative of the knowledge of a person of skill in the art and illustrate other aspects of the present invention as envisioned by the inventors.

### REFERENCE LIST

Chen, P.-J., Kalpana, G., Goldberg, J., 1986. Structure and Replication of the Genome of the Hepatitis 6 Virus. Proceedings of the National Academy of Sciences 83(22), 8774-8778.
Ferns, R., Nastouli, E., Garson, J., 2012. Quantitation of hepatitis delta virus using a single-step internally controlled real-time RT-qPCR and full-length genomic RNA calibration standard. Journal of Virological Methods 179, 189-194.
Hadler, S. C., De Monzon, M., Ponzetto, A., Anzola, E., Rivera, D., Mondolfi, A; Bracho, A., Francis, D., Gerber, M., Thung, S., Gerin, J., Maynard, J., Popper, H., and Purcell, H., 1984. Delta virus infection and severe hepatitis. An epidemic in the Yucpa Indians of Venezuela. Ann. Internal Med. 100, 339-344.
Hoffman, J., Frenzel, K., Minh, B., von Haeseler, A., Edelmann, A., Ross, S., Berg, T., Krüger, D. and Meisel, H., 2010. Quantitative detection and typing of hepatitis D virus in human serum by real-time polymerase chain reaction and melting curve analysis 67, 172-179.
Hughes, S. A., Wedemeyer, H., Harrison, P., 2011. Hepatitis delta virus. Lancet, 378, 73-85.

Kodani, M., Winchell, J., 2012. Engineered Combined-Positive-Control Template for Real-Time Reverse Transcription-PCR in Multiple-Pathogen-Detection Assays. Journal of Clinical Microbiology 50(3), 1057-1060.
Lai, M. M. C., 2005. RNA replication without RNA-dependent RNA polymerase: surprises from hepatitis delta virus. Journal of Virology 79, 7951-7958.
Le Gal, F., Gault, E., Ripault, M.-P., Serpaggi, J., Trinchet, J.-C., Gordien, E., Deny, P., 2006. Eight Major Clade for Hepatitis Delta Virus. Emerging Infectious Diseases 12(9), 1447-1450.
Le Gal, F., Gordien, E., Affolabi, D., Hanslik, T., Alloui, C., Deny, P., Gault, E., 2005. Quantification of Hepatitis Delta Virus RNA in Serum by Concensus Real-Time PCR Indicates Different Patterns of Virological Response to Interferon Therapy in Chronically Infected Patients. Journal of Clinical Microbiology 43(5), 2363-2369.
Mason, W., Burrell, C., Casey, J., 2005. Deltavirus. In: Fauquet, C., Mayo, M., Maniloff, J., Desselberger, U., Ball, L., eds. Eight Report of the International Committee on Taxonomy of Viruses. London: Elsevicr/Academic Press. 735-738.
Mederacke, I., Bremer, B., Heidrich, B., Kirschner, J., Deterding, K., Bock, T., Wursthorn, M., Manns, P., Wedemeyer, H., 2010. Establishment of a Novel Quantitative Hepatitis D Virus (HDV) RNA Assay Using the Cobas TaqMan Platform to Study HDV RNA Kinetics. J Clin Micro 48(6), 2022-2029.
Pascarella, S., Negro, F., 2010. Hepatitis D Virus: an Update. Liver International, 7-21.
Radjef N., Gordien, E., Ivaniushina, V., Gault, E., Anais, P., Drugan, T., 2004. Molecular phylogenetic analyses indicate a wide and ancient radiation African hepatitis delta virus, suggesting a deltavirus genus of at least seven major clades. J Virol 78, 2537-2544.
Rizzetto, M., Hoyer, B., Canese, M. G., Shih, J. W. K., Purcell, R. H., Gerin, J. L., 1980. Delta agent: association of delta antigen with hepatitis B surface antigen and RNA in serum of delta-infected chimpanzees. Proc Natl Acad Sci USA 77, 6124-6128.
Scholtes, C., Icard, V., Amiri, M., Chevallier-Queyron, P., Trabaud, M., Ramiere, C., Zoulim, F., Andre, P., Deny, P., 2012. Standardized One-Step Real-Time Reverse Transcription-PCR Assay for Universal Detection and Quantification of Hepatitis Delta Virus from Clinical Samples in the Presence of a Heterologous Internal-Control RNA. J Clin Micro 50(6), 2126-2128.
Shang, D., Hughes S., Homer, M., Bruce M., Dong. Y., Carey, I., Suddle, A., Agrawal, K., Harrison, P., and Atkins M., 2012. Development and validation of an efficient in-house real-time reverse transcription polymerase chain reaction assay for the quantitative detection of serum hepatitis delta virus RNA in a diverse South London population. J Virol Met 184, 55-62.
Sharmeen L, Kuo M Y, Dinter-Gottlieb G, Taylor J., 1988. Antigenomic RNA of Human Hepatitis Delta Virus Can Undergo Self-Cleavage. J Virol. 62, 2674-2679.
Wang, K., Choo, Q., Weiner, A., et al. 1986. Structure, sequence and expression of the hepatitis delta (delta) viral genome. Nature 323, 508-514.
Wedemeyer, H., Manns, M. P., 2010. Epidemiology, pathogenesis and management of hepatitis D: update and challenges ahead. Nat Rev Gastro Hep 7, 31-40.
Wu, H., Lin, Y., Lin F P, Makino S, Chang M F, Lai M M, 1989. Human Hepatitis Delta Virus RNA Subfragments Contain an Autocleavage aActivity. Proc Natl Acad Sci USA 86, 1831-1835.
Martin A, Lemon S M. 2006. Hepatitis A virus: from discovery to vaccines. Hepatology 43:S164-172.
Kao J H. 2011. Molecular epidemiology of hepatitis B virus. The Korean journal of internal medicine 26:255-261.
Hajarizadeh B, Grebely J, Dore G J. 2013. Epidemiology and natural history of HCV infection. Nature reviews. Gastroenterology & hepatology.
Ward J W. 2013. The epidemiology of chronic hepatitis C and one-time hepatitis C virus testing of persons born during 1945 to 1965 in the United States. Clinics in liver disease 17:1-11.
Kim C W, Chang K M. 2013. Hepatitis C virus: virology and life cycle. Clinical and molecular hepatology 19:17-25.
Niro G A, Smedile A. 2012. Current concept in the pathophysiology of hepatitis delta infection. Current infectious disease reports 14:9-14.
Hughes S A, Wedemeyer H, Harrison P M. 2011. Hepatitis delta virus. Lancet 378:73-85.
Aggarwal R, Jameel S. 2011. Hepatitis E. Hepatology 54:2218-2226.
Panda S K, Thakral D, Rehman S. 2007. Hepatitis E virus. Reviews in medical virology 17:151-180.
Kodani M, Yang G, Conklin L M, Travis T C, Whitney C G, Anderson L J, Schrag S J, Taylor T H, Jr., Beall B W, Breiman R F, Feikin D R, Njcnga M K, Mayer L W, Oberste M S, Tondella M L, Winchell J M, Lindstrom S L, Erdman D D, Fields B S. 2011. Application of TaqMan low-density arrays for simultaneous detection of multiple respiratory pathogens. Journal of clinical microbiology 49:2175-2182.
Weinberg G A, Schnabel K C, Erdman D D, Prill M M, Iwane M K, Shelley L M, Whitaker B L, Szilagyi P G, Hall C B. 2013. Field evaluation of TaqMan Array Card (TAC) for the simultaneous detection of multiple respiratory viruses in children with acute respiratory infection. Journal of clinical virology: the official publication of the Pan American Society for Clinical Virology 57:254-260.
Diaz M H, Waller J L, Napoliello R A, Islam M S, Wolff B J, Burken D J, Holden R L, Srinivasan V, Arvay M, McGee L, Oberste M S, Whitney C G, Schrag S J, Winchell J M, Saha S K. 2013. Optimization of Multiple Pathogen Detection Using the TaqMan Array Card: Application for a Population-Based Study of Neonatal Infection. PloS one 8:e66183.
Liu J, Gratz J, Amour C, Kibiki G, Becker S, Janaki L, Verweij J J, Taniuchi M, Sobuz S U, Haque R, Haverstick D M, Houpt E R. 2013. A laboratory-developed TaqMan Array Card for simultaneous detection of 19 enteropathogens. Journal of clinical microbiology 51:472-480.
Rachwal P A, Rose H L, Cox V, Lukaszewski R A, Murch A L, Weller S A. 2012. The potential of TaqMan Array Cards for detection of multiple biological agents by real-time PCR. PloS one 7:e35971.
Weller S A, Cox V, Essex-Lopresti A, Hartley M G, Parsons T M, Rachwal P A, Stapleton H L, Lukaszewski R A. 2012. Evaluation of two multiplex real-time PCR screening capabilities for the detection of Bacillus anthracis, Francisella tularensis and Yersinia pestis in blood samples generated from murine infection models. Journal of medical microbiology 61:1546-1555.
Garson J A, Ferns R B, Grant P R, Ijaz S, Nastouli E, Szypulska R, Tedder R S. 2012. Minor groove binder modification of widely used TaqMan probe for hepatitis E virus reduces risk of false negative real-time PCR results. Journal of virological methods 186:157-160.
Jothikumar N, Cromeans T L. Robertson B H, Meng X J, Hill V R. 2006. A broadly reactive one-step real-time R T-PCR assay for rapid and sensitive detection of hepatitis E virus. Journal of virological methods 131:65-71.
Kodani M, Martin A, Mixson-Hayden T, Drobeniuc J, Gish R R, Kamili S. 2013. One-step real-time PCR assay for detection and quantitation of hepatitis D virus RNA. Journal of virological methods.
Altschul S F, Gertz E M, Agarwala R, Schaffer A A, Yu Y K. 2009. PSI-BLAST pseudocounts and the minimum description length principle. Nucleic acids research 37:815-824.
Kodani M, Winchell J M. 2012. Engineered combined-positive-control template for real-time reverse transcription-PCR in multiple-pathogen-detection assays. Journal of clinical microbiology 50:1057-1060.
Nainan O V, Xia G, Vaughan G, Margolis H S. 2006. Diagnosis of hepatitis a virus infection: a molecular approach. Clinical microbiology reviews 19:63-79.

1. A process of detecting hepatitis delta virus (HDV) in a biological sample comprising:
producing an amplification product by amplifying a HDV nucleotide sequence using a forward primer that interacts with an HDV genome at positions 816-834, and a reverse primer, under conditions suitable for a polymerase chain reaction; and
measuring said amplification product to detect HDV in said biological sample.

2. The process of claim 1 where said forward primer includes at least two degenerate nucleotide positions.

3. The process of claim 1 where said forward primer consists of the sequence of SEQ ID NO: 1.

4. The process of claim 1 where said reverse primer binds a region of a HDV genome between nucleotides 894 to 908.

5. The process of claim 1 where said reverse primer consists of the sequence of SEQ ID NO: 2.

6. The process of claim 1 where said measuring is by using a probe that will bind a region of a HDV genome in the region of nucleotides 853 to 890.

7. The process of claim 6 where said probe includes at least two positions of nucleotide degeneracy.

8. The process of claim 6 where said probe consists of the nucleotide sequence of SEQ ID NO: 3.

9-15. (canceled)

16. The process of claim 1 further comprising
producing a second amplification product by amplifying a nucleotide sequence from one or more second viruses selected from the group consisting of HAV, HBV, HCV, and HEV; and
measuring said second amplification product to detect said virus in said biological sample.

17. The process of claim 16 wherein:
i. said second virus is HAV and said step of producing is using one or more nucleotides selected from the group consisting of SEQ ID NO: 16, SEQ ID NO: 17, and SEQ ID NO: 18;
ii. said second virus is HBV and said step of producing is using one or more nucleotides selected from the group consisting of SEQ ID NO: 19, SEQ ID NO: 20, and SEQ ID NO: 21;
iii. said second virus is HCV and said step of producing is using one or more nucleotides selected from the group consisting of SEQ ID NO: 22, SEQ ID NO: 23, and SEQ ID NO: 24;
iv. said second virus is HEV and said step of producing is using one or more nucleotides selected from the group consisting of SEQ ID NO: 25, SEQ ID NO: 26, and SEQ ID NO: 27;
or combinations thereof.

18-20. (canceled)

21. A process of detecting the presence or absence of one or more hepatitis viruses selected from the group consisting of HAV, HBV, HCV, HDV, and HEV in a biological sample comprising:
producing an amplification product by amplifying a hepatitis virus nucleotide sequence using a forward primer and a reverse primer, under conditions suitable for a polymerase chain reaction wherein reaction conditions for said step of producing HAV, HBV, HCV, HDV, and HDV are identical, wherein said forward primer comprises a nucleotide sequence selected from the group consisting of SEQ ID NO: 1, SEQ ID NO: 16, SEQ ID NO: 19, SEQ ID NO: 22, SEQ ID NO: 25, and combinations of said primers; and
measuring said amplification product to detect one or more hepatitis viruses in said biological sample.

22-25. (canceled)

26. The process according to claim 21 wherein said step of measuring comprises using a probe comprising a nucleotide sequence selected from the group consisting of SEQ ID NO: 3, SEQ ID NO: 6, SEQ ID NO: 7, SEQ ID NO: 8, SEQ ID NO: 9, SEQ ID NO: 10, SEQ ID NO: 18, SEQ ID NO: 21, SEQ ID NO: 24, SEQ ID NO: 27, and combinations of said probes.

27. The process of claim 21 where said detecting diagnoses hepatitis virus infection in a subject.

28. A kit for detecting hepatitis delta virus infection comprising a first forward primer, said first forward primer optionally interacts with an HDV genome at positions 816-834; and
one or more ancillary reagents.

29. The kit of claim 28 wherein said first forward primer includes at least two degenerate nucleotides.

30. The kit of claim 28 where said forward primer consists of the sequence of SEQ ID NO: 1, SEQ ID NO: 16, SEQ ID NO: 19, SEQ ID NO: 22, or SEQ ID NO: 25, or combinations of said primers.

31-32. (canceled)

33. The kit of claim 28 further comprising a probe that will bind a region of a HDV genome in the region of nucleotides 853 to 890.

34. The kit of claim 33 in which said probe comprises the nucleotide sequence SEQ ID NO: 3, SEQ ID NO: 6, SEQ ID NO: 7, SEQ ID NO: 8, SEQ ID NO: 9, SEQ ID NO: 10, SEQ ID NO: 18, SEQ ID NO: 21, SEQ ID NO: 24, SEQ ID NO: 27, and combinations of said probes.

35-37. (canceled)

38. An oligonucleotide comprising consisting of: the sequence SEQ ID NO: 1; SEQ ID NO: 2; SEQ ID NO: 4, SEQ ID NO: 5; SEQ ID NO: 16; SEQ ID NO: 17; SEQ ID NO: 19; SEQ ID NO: 20; SEQ ID NO: 22; SEQ ID NO: 23; SEQ ID NO: 25; or SEQ ID NO: 26.

39-77. (canceled)

78. An oligonucleotide comprising a label and the sequence; SEQ ID NO: 3; SEQ ID NO: 6, SEQ ID NO: 7; SEQ ID NO: 8; SEQ ID NO: 9; SEQ ID NO: 10; SEQ ID NO: 18; SEQ ID NO: 21; SEQ ID NO: 24; or SEQ ID NO: 27.


SEQUENCE LISTING (27 sequences)

SEQ ID NO: 1 - DNA, 19 bp, Hepatitis D virus
tctcccttwgccatcmgag

SEQ ID NO: 2 - DNA, 15 bp, Hepatitis D virus
tcctcttcgggtcgg

SEQ ID NO: 3 - DNA, 19 bp, Hepatitis D virus
cycgcggtccgwcctgggc

SEQ ID NO: 4 - DNA, 16 bp, Hepatitis D virus
tcctcttcgggtcggc

SEQ ID NO: 5 - DNA, 19 bp, Hepatitis D virus
tcctcttcgggtcggcatg

SEQ ID NO: 6 - DNA, 23 bp, Hepatitis D virus
cycgcggtccgwcctggcrtccg

SEQ ID NO: 7 - DNA, 23 bp, Hepatitis D virus
cctccycgcggtccgwcctgggc

SEQ ID NO: 8 - DNA, 22 bp, Hepatitis D virus
ctccycgcggtccgwcctgggc

SEQ ID NO: 9 - DNA, 21 bp, Hepatitis D virus
tccycgcggtccgwcctgggc

SEQ ID NO: 10 - DNA, 20 bp, Hepatitis D virus
ccycgcggtccgwcctgggc

SEQ ID NO: 11 - DNA, 18 bp, Hepatitis D virus
agaaagcaacggggctag

SEQ ID NO: 12 - DNA, 18 bp, Hepatitis D virus
ttcctcttcgggtcggca

SEQ ID NO: 13 - DNA, 18 bp, Hepatitis D virus
tctttcctcttcgggtcg

SEQ ID NO: 14 - DNA, 18 bp, Hepatitis D virus
tgccgagtgaggcttatc

SEQ ID NO: 15 - DNA, 18 bp, Hepatitis D virus
gaggaggctgggaccatg

SEQ ID NO: 16 - DNA, 24 bp, Hepatitis A virus
gggtgaaacctcttaggctaatac

SEQ ID NO: 17 - DNA, 17 bp, Hepatitis A virus
tcctccggcgttgaatg

SEQ ID NO: 18 - DNA, 30 bp, Hepatitis A virus
caccaatatccgccgctgttaccctatcca

SEQ ID NO: 19 - DNA, 20 bp, Hepatitis B virus
tgtcctggytatcgctggat

SEQ ID NO: 20 - DNA, 21 bp, Hepatitis B virus
aagaaccaayaagaagatgag

SEQ ID NO: 21 - DNA, 28 bp, Hepatitis B virus
tgcggcgttttatcatmttcctcttcat

SEQ ID NO: 22 - DNA, 20 bp, Hepatitis C virus
ctagccgagtagygttgggt

SEQ ID NO: 23 - DNA, 20 bp, Hepatitis C virus
catgttgcacggtctacgag

SEQ ID NO: 24 - DNA, 26 bp, Hepatitis C virus
ctcgcaagcaccctatcaggcagtac

SEQ ID NO: 25 - DNA, 18 bp, Hepatitis E virus
ggtggtttctggggtgac

SEQ ID NO: 26 - DNA, 18 bp, Hepatitis E virus
aggggttggttggatgaa

SEQ ID NO: 27 - DNA, 18 bp, Hepatitis E virus
tgattctcagcccttcgc